(19) United States
(12) Patent Application Publication
(10) Pub. No.: US 2025/0099240 A1
(43) Pub. Date: Mar. 27, 2025

(54) REINFORCEMENT MEMBER FOR AN OUTER SKIRT OF A PROSTHETIC HEART VALVE

(71) Applicant: EDWARDS LIFESCIENCES CORPORATION, Irvine, CA (US)

(72) Inventor: David Maimon, Atlit (IL)

(21) Appl. No.: 18/975,500

(22) Filed: Dec. 10, 2024

Related U.S. Application Data
(63) Continuation of application No. PCT/US2023/025823, filed on Jun. 21, 2023.
(60) Provisional application No. 63/366,799, filed on Jun. 22, 2022.

(51) Int. Cl. A61F 2/24

(52) U.S. Cl. CPC A61F 2/2418; A61F 2210/0057

(57) ABSTRACT

Reinforcement members for an outer skirt of a prosthetic heart valve are disclosed. As one example, a prosthetic heart valve can include an annular frame and an outer skirt disposed around an outer surface of the frame, the outer skirt including a sealing layer comprising an outflow edge portion secured to the frame and an inflow edge portion secured to the frame and a deformable reinforcement member attached to the sealing layer. The reinforcement member extends circumferentially along the sealing layer and is configured to extend radially outward from the frame such that the sealing layer bulges radially outward and away from the frame when the frame is radially expanded from a radially compressed state to a radially expanded state. The reinforcement member comprises a circumferentially extending row of cells or an undulating shape.

### CROSS-REFERENCE TO RELATED APPLICATIONS

This application is a continuation of PCT patent application no. PCT/US2023/025823 filed on Jun. 21, 2023, which application claims the benefit of U.S. Provisional Patent Application No. 63/366,799, filed Jun. 22, 2022, each of these applications being incorporated by reference herein in its entirety.

### FIELD

The present disclosure relates to prosthetic heart valves, and in particular to outer coverings or skirts with deformable sealing members for prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

Most expandable, prosthetic heart valves comprise a cylindrical metal frame or stent and prosthetic leaflets mounted inside the frame. These valves can also include one or more coverings (or skirts) spanning a circumference of the frame, on an inner or outer surface of the frame. These coverings can be configured to establish a seal with the native tissue when the prosthetic heart valve is placed at the implantation site (and thus may be referred to as sealing members). However, the native tissue (e.g., at the native valve annulus or arterial wall around the native valve) can have an irregular shape while the frame of the prosthetic heart valve is generally cylindrical. As a result, gaps can be formed between the prosthetic heart valve and native heart valve annulus when the prosthetic heart valve is implanted within the native heart valve annulus, even when coverings are included on the prosthetic heart valve.

Accordingly, a need exists for improved coverings or outer skirts for prosthetic heart valves which can better fills gaps between the native tissue and the prosthetic heart valve.

### SUMMARY

Described herein are prosthetic heart valves, delivery apparatus, and methods for implanting prosthetic heart valves. In particular, described herein are examples of coverings for a prosthetic heart valve and methods of making and using such coverings. Prosthetic heart valves can include a frame and a leaflet assembly arranged on an inner surface of the frame. The prosthetic heart valve can include a covering (or outer skirt) arranged around a circumference of the frame and on an outer surface of the frame. The outer skirt can include one or more reinforcement members (or sealing members) that are deformable (or pliable) and configured to bulge radially outward and away from the frame. In some examples, the one or more reinforcement members can extend circumferentially around the frame. The one or more reinforcement members can be configured such that the prosthetic heart valve, once implanted, better conforms to a shape of the surrounding native tissue. As such, the skirts and prosthetic heart valves disclosed herein can, among other things, overcome one or more of the deficiencies of typical prosthetic heart valves.

A prosthetic heart valve can comprise a frame and a valvular structure coupled to the frame. In addition to these components, a prosthetic heart valve can further comprise one or more of the components disclosed herein.

In some examples, the prosthetic heart valve can comprise a sealing member configured to reduce paravalvular leakage.

A prosthetic heart valve can comprise a frame and a sealing member configured to reduce paravalvular leakage. In addition to these components, a prosthetic heart valve can further comprise one or more of the components disclosed herein.

In some examples, the sealing member is an outer skirt disposed around an outer surface of the frame.

In some examples, the outer skirt can comprise a sealing layer comprising an outflow edge portion secured to the frame and an inflow edge portion secured to the frame; and a deformable reinforcement member attached to the sealing layer.

In some examples, the reinforcement member extends circumferentially along the sealing layer.

In some examples, the reinforcement member can comprise a circumferentially extending row of cells or an undulating shape.

In some examples, the reinforcement member can comprise a shape-memory material and is moveable between a first shape and a second shape.

In some examples, the reinforcement member can comprise a circumferentially extending row of cells.

In some examples, a prosthetic heart valve comprises: an annular frame and an outer skirt disposed around an outer surface of the frame. The outer skirt comprises: a sealing layer comprising an outflow edge portion secured to the frame and an inflow edge portion secured to the frame; and a deformable reinforcement member attached to the sealing layer, the reinforcement member extending circumferentially along the sealing layer. The reinforcement member is configured to extend radially outward from the frame such that the sealing layer bulges radially outward and away from the frame when the frame is radially expanded from a radially compressed state to a radially expanded state. The reinforcement member comprises a circumferentially extending row of cells or an undulating shape.

In some examples, a prosthetic heart valve comprises: a radially expandable and compressible annular frame configured to move between a radially compressed configuration and a radially expanded configuration; and an outer skirt disposed around an outer surface of the frame. The outer skirt comprises a sealing layer comprising an outflow edge portion secured to the frame and an inflow edge portion secured to an inflow end of the frame, and a reinforcement member coupled to the sealing layer and extending circumferentially along the sealing layer at an axial location that is between the outflow edge portion and the inflow edge portion. The reinforcement member comprises a shape-memory material and is moveable between a first shape and a second shape, where in the first shape the reinforcement member is lengthened in an axial direction and disposed closer to the frame relative to the second shape, and where in second shape the reinforcement member is shortened in an axial direction relative to the first shape and protrudes radially outward and away from the frame.

In some examples, a prosthetic heart valve comprises: an annular frame and an outer skirt disposed around an outer surface of the frame. The outer skirt comprises a sealing layer disposed around an outer surface of the frame, and a deformable reinforcement member coupled to the sealing layer, the reinforcement member extending circumferentially along the sealing layer and comprising a circumferentially extending row of cells.

In some examples, a prosthetic heart valve comprises: an annular frame and an outer skirt disposed around an outer surface of the frame. The outer skirt comprises a sealing layer disposed around an outer surface of the frame, and a shape-memory reinforcement wire comprising a circumferentially extending row of cells supported by the sealing layer.

In some examples, a prosthetic heart valve comprises an annular frame, and an outer skirt disposed around an outer surface of the frame. The outer skirt comprises a sealing layer disposed around an outer surface of the frame, and a deformable reinforcement member coupled to the sealing layer, the reinforcement member extending circumferentially along the sealing layer and comprising a plurality of axially extending members that are spaced circumferentially apart from one another around the outer skirt.

In some examples, a prosthetic heart valve comprises one or more of the components recited in Examples 1-50, 57-64, and 66-68 below.

A method can comprise radially expanding a prosthetic valve from a radially compressed configuration to a radially expanded configuration within a native anatomy, where the prosthetic heart valve comprises an annular frame and an outer skirt disposed around an outer surface of the frame comprising a sealing layer secured to the frame. The method can further comprise, during the radially expanding, transitioning a reinforcement member embedded within a portion of the sealing layer that is unattached to the frame from a first configuration to a second configuration, where in the first configuration the reinforcement member is disposed adjacent to the frame, and where in the second configuration the reinforcement member extends radially outward and away from the frame.

In some examples, a method comprises radially expanding a prosthetic heart valve from a radially compressed configuration to a radially expanded configuration within a native valve annulus of a heart, where the prosthetic heart valve comprises an annular frame, a plurality of leaflets secured inside the frame, and an outer skirt disposed around an outer surface of the frame comprising a sealing layer secured to the frame at an outflow edge portion of the sealing layer and an inflow edge portion of the sealing layer. The method further comprises, during the radially expanding, transitioning a reinforcement member embedded within a portion of the sealing layer that is unattached to the frame from a first configuration to a second configuration, where in the first configuration the reinforcement member is disposed adjacent to the frame and is longer in an axial direction relative to the second configuration, and where in the second configuration the reinforcement member extends radially outward and away from the frame.

The above method(s) can be performed on a living animal or on a simulation, such as on a cadaver, cadaver heart, anthropomorphic ghost, simulator (e.g., with body parts, heart, tissue, etc. being simulated).

In some examples, a method comprises one or more of the features recited in Examples 51-56 and 65 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a perspective view of a prosthetic heart valve, according to one example.

FIG. 2 is a perspective view of a delivery apparatus for a prosthetic heart valve, according to an example.

FIG. 3 is a side view of an outer skirt for a prosthetic heart valve, according to one example, the outer skirt shown in a flattened configuration and including a reinforcement member.

FIG. 4A is a schematic illustrating the outer skirt of FIG. 3 arranged around an exemplary frame of a prosthetic device, the frame in a radially compressed configuration and the reinforcement member in a first configuration.

FIG. 4B is a schematic illustrating the outer skirt of FIG. 3 arranged around the frame of a prosthetic device, the frame in a radially expanded configuration and the reinforcement member in a second configuration.

FIG. 5 is a schematic illustrating a prosthetic device implanted (radially expanded) within an exemplary native valve annulus having a depression therein, the prosthetic device including the outer skirt of FIG. 3 which extends into the depression of the native valve annulus.

FIG. 6 is a schematic illustrating a prosthetic device implanted (radially expanded) within an exemplary native valve annulus including calcified nodules, the prosthetic device including the outer skirt of FIG. 3 which extends into depressions of the native valve annulus disposed adjacent to the calcified nodules.

FIG. 7 is a side view of an outer skirt for a prosthetic heart valve, according to another example, the outer skirt shown in a flattened configuration and including reinforcement members shaped as horizontally extending lines along the outer skirt.

FIG. 8 is a side view of an outer skirt for a prosthetic heart valve, according to another example, the outer skirt shown in a flattened configuration and including a reinforcement member forming a zig-zag pattern along the outer skirt.

FIG. 9 is a side view of an outer skirt for a prosthetic heart valve, according to another example, the outer skirt shown in a flattened configuration and including reinforcement members shaped as axially extending rectangles that are spaced apart from one another along the outer skirt.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, “e.g.” means “for example,” and “i.e.” means “that is.”

### Overview of the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient's vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

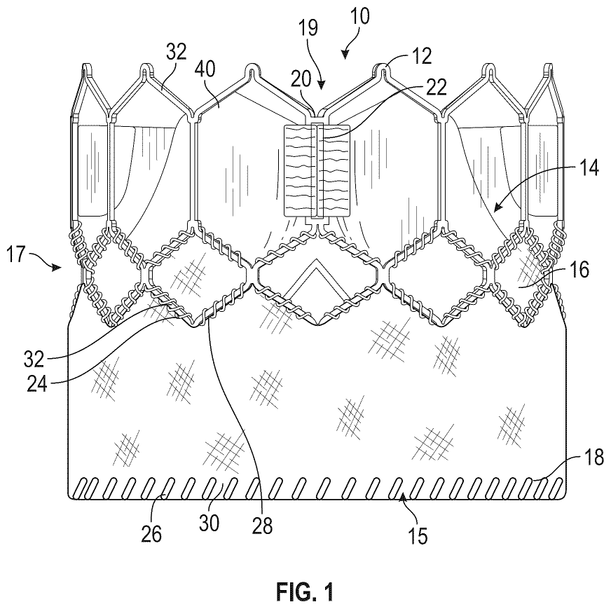

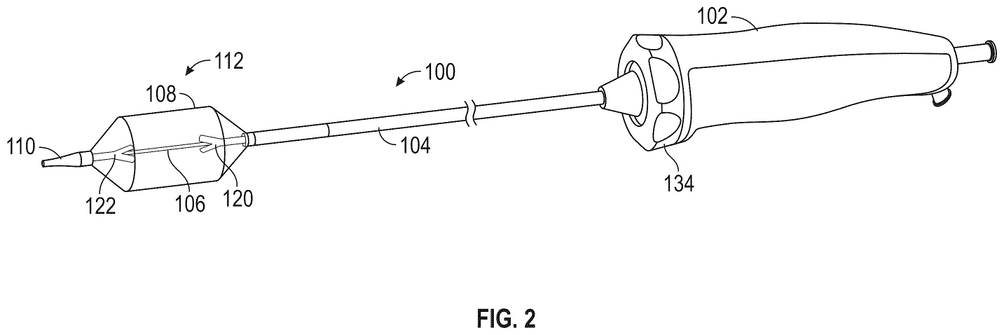

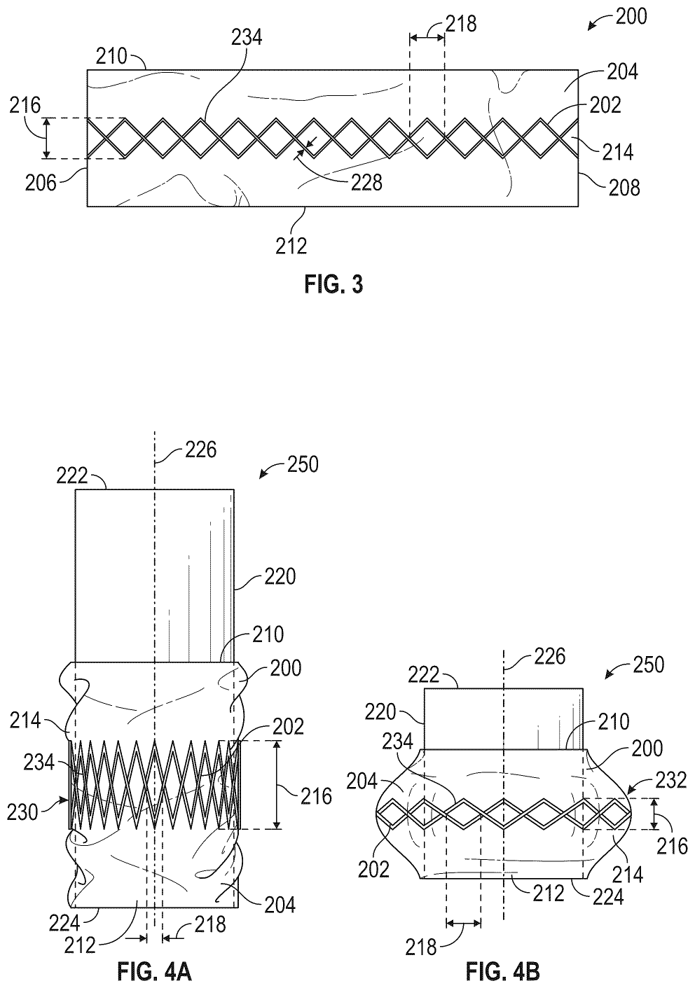

FIG. 1 illustrates an exemplary prosthetic device (e.g., prosthetic heart valve) that can be advanced through a patient's vasculature, such as to a native heart valve, by a delivery apparatus, such as the exemplary delivery apparatus shown in FIG. 2. The prosthetic heart valve can include an outer covering or skirt disposed around an outer surface of the frame of the prosthetic heart valve. The outer skirt can comprise one or more sealing or reinforcement members that are deformable and bulge radially outward from the frame. Thus, such outer skirts are configured to improve sealing of the prosthetic heart valve against native tissue at an implantation site. Various examples of deformable reinforcement or sealing members for outer skirts and methods of forming such reinforcement members are shown in FIGS. 3-9.

### EXAMPLES OF THE DISCLOSED TECHNOLOGY

FIG. 1 shows an exemplary prosthetic valve 10, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic valve 10 can have four main components: a stent or frame 12, a valvular structure 14, an inner skirt 16, and a perivalvular outer sealing member or outer skirt 18. The prosthetic valve 10 can have an inflow end portion 15, an intermediate portion 17, and an outflow end portion 19.

The valvular structure 14 can comprise three leaflets 40, collectively forming a leaflet structure, which can be arranged to collapse in a tricuspid arrangement, although in other examples there can be greater or fewer number of leaflets (e.g., one or more leaflets 40). The leaflets 40 can be secured to one another at their adjacent sides to form commissures 22 of the valvular (e.g., leaflet) structure 14. The lower edge of valvular structure 14 can have an undulating, curved scalloped shape and can be secured to the inner skirt 16 by sutures (not shown). In some examples, the leaflets 40 can be formed of pericardial tissue (e.g., bovine pericardial tissue), biocompatible synthetic materials, or various other suitable natural or synthetic materials as known in the art and described in U.S. Pat. No. 6,730,118, which is incorporated by reference herein.

The frame 12 can be formed with a plurality of circumferentially spaced slots, or commissure windows 20 that are adapted to mount the commissures 22 of the valvular structure 14 to the frame. The frame 12 can be made of any of various suitable plastically-expandable materials (e.g., stainless steel, etc.) or self-expanding materials (e.g., nickel titanium alloy (NiTi), such as nitinol), as known in the art. When constructed of a plastically-expandable material, the frame 12 (and thus the prosthetic valve 10) can be crimped to a radially collapsed (or compressed) configuration on a delivery catheter and then expanded inside a patient by an inflatable balloon or equivalent expansion mechanism to a radially expanded configuration. When constructed of a self-expandable material, the frame 12 (and thus the prosthetic valve 10) can be crimped to a radially collapsed configuration and restrained in the collapsed configuration by insertion into a sheath or equivalent mechanism of a delivery catheter. Once inside the body, the prosthetic valve can be advanced from the delivery sheath, which allows the prosthetic valve to expand to its functional size.

Suitable plastically-expandable materials that can be used to form the frame 12 include metal alloys, polymers, or combinations thereof. Example metal alloys can comprise one or more of the following: nickel, cobalt, chromium, molybdenum, titanium, or other biocompatible metal. In some examples, the frame 12 can comprise stainless steel. In some examples, the frame 12 can comprise cobalt-chromium. In some examples, the frame 12 can comprise nickel-cobalt-chromium. In some examples, the frame 12 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

Additional details regarding the prosthetic valve 10 and its various components are described in WIPO Patent Application Publication No. WO 2018/222799, which is incorporated herein by reference.

The frame 12 can comprise a plurality of interconnected struts 32 that form open cells in the frame.

In some examples, as shown in FIG. 1 an upper edge portion 28 (also referred to as an outflow edge portion) of the outer skirt 18 can be secured to the frame 12 by stitches 24 and a lower edge portion 30 (also referred to as an inflow edge portion) of the outer skirt 18 can be secured to the frame 12 by stitches 26 extending along the inflow end portion 15 of the prosthetic valve 10. For example, the stitches 24 can wrap around struts 32 of the frame 12 forming a row of circumferentially extending struts 32 at the intermediate portion 17 of the prosthetic valve 10. Further, in some examples, the stitches 26 can wrap around struts 32 of the frame 12 forming a row of circumferentially extending struts 32 at the inflow end portion 15 of the prosthetic valve 10.

FIG. 2 shows a delivery apparatus 100, according to an example, that can be used to implant an expandable prosthetic heart valve (e.g., prosthetic valve 10), or another type of expandable prosthetic medical device (such as a stent). In some examples, the delivery apparatus 100 is specifically adapted for use in introducing a prosthetic valve into a heart.

The delivery apparatus 100 in the illustrated example of FIG. 2 is a balloon catheter comprising a handle 102, a steerable, outer shaft 104 extending from the handle 102, an intermediate shaft extending from the handle 102 coaxially through the steerable outer shaft 104, and an inner shaft 106 extending from the handle 102 coaxially through the intermediate shaft and the steerable, outer shaft 104, an inflatable balloon (e.g., balloon) 108 extending from a distal end of the intermediate shaft, and a nosecone 110 arranged at a distal end of the delivery apparatus 100. A distal end portion 112 of the delivery apparatus 100 includes the balloon 108, the nosecone 110, and a balloon shoulder assembly. A prosthetic medical device, such as a prosthetic heart valve may be mounted on a valve retaining portion of the balloon 108. A balloon shoulder assembly is configured to maintain the prosthetic heart valve or other medical device at a fixed position on the balloon 108 during delivery through the patient's vasculature. In some examples, the balloon shoulder assembly can include a proximal shoulder 120 and/or a distal shoulder 122.

The balloon 108 can include a central portion (which can be approximately cylindrical when inflated, as shown in FIG. 2) and two tapered end portions that connect to the delivery apparatus 100 (e.g., to one or more shafts and/or a nosecone of the delivery apparatus).

The handle 102 can include a steering mechanism configured to adjust the curvature of the distal end portion of the delivery apparatus. In the illustrated example, for example, the handle 102 includes an adjustment member, such as the illustrated rotatable knob 134, which in turn is operatively coupled to the proximal end portion of a pull wire (not shown). The pull wire extends distally from the handle 102 through the outer shaft 104 and has a distal end portion affixed to the outer shaft at or near the distal end of the outer shaft 104. Rotating the knob 134 is effective to increase or decrease the tension in the pull wire, thereby adjusting the curvature of the distal end portion of the delivery apparatus.

The delivery apparatus 100 can be configured to be advanced over a guidewire that can be received within a guidewire lumen defined by an innermost shaft of the delivery apparatus 100.

In some examples, the delivery apparatus (or another, similar delivery apparatus) can be configured to deploy and implant a prosthetic heart valve (e.g., prosthetic valve 10 of FIG. 1) in the native aortic annulus of a native aortic valve. Further details on such a delivery apparatus can be found in International Application No. PCT/US2021/047056, which in incorporated by reference herein.

As an example, during an implantation procedure for implanting an expandable prosthetic heart valve (e.g., prosthetic valve 10 of FIG. 1), the distal end portion of the delivery apparatus 100 (or another similar delivery apparatus or balloon catheter) can be advanced (over a guidewire) to a target implantation site (e.g., a native valve annulus). The balloon 108 can then be inflated to radially expand and implant the prosthetic heart valve within the native valve annulus.

The native valve annulus and/or surrounding anatomy of the implantation site (such as the arterial wall) can have an irregular shape that can include depressed regions, calcified nodules, and/or alternate protrusions. However, since the prosthetic heart valve can have a generally cylindrical shape, when the prosthetic heart valve is implanted within the native valve annulus, gaps (e.g., voids or channels) can be formed between the prosthetic heart valve and the native valve annulus. Such gaps can increase a likelihood of perivalvular leakage past the prosthetic heart valve.

Thus, it may be desirable to provide an outer skirt with a reinforcement member that is configured to extend or bulge radially outward from a frame of the prosthetic heart valve and into depressed regions of the native valve annulus, thereby providing increased paravalvular leakage sealing against the native valve annulus.

FIGS. 3-4B depict one example of an outer skirt 200 (also referred to as a sealing member) for a prosthetic device 250 (such as prosthetic heart valve 10 of FIG. 1) including a reinforcement member 202 configured to extend radially outward from a frame 220 of the prosthetic device 250 when the prosthetic device 250 is radially expanded (e.g., when implanted at a native valve annulus or alternate implantation site). The outer skirt 200, as well as the other outer skirts disclosed herein, can be used in a mechanically expandable prosthetic valve, a balloon-expandable prosthetic valve (e.g., prosthetic valve 10 of FIG. 1), and/or a self-expandable prosthetic valve. Additional details on balloon expandable prosthetic valves can be found in U.S. Pat. No. 9,393,110, and U.S. Provisional Application Nos. 63/178,416, filed Apr. 22, 2021, 63/194,830, filed May 28, 2021, and 63/279,096, filed Nov. 13, 2021, all of which are incorporated by reference herein. Additional details on a mechanically expandable prosthetic valve can be found in International Application PCT/US2021/052745, filed Sep. 30, 2021, which is incorporated by reference herein. Additional details on a self-expanding prosthetic valve can be found in U.S. Pat. No. 8,652,202, which is incorporated by reference herein.

FIG. 3 shows the outer skirt 200 in a flattened configuration and FIGS. 4A and 4B show the outer skirt 200 in an annular configuration, arranged around and secured to an outer surface of a frame 220 of a prosthetic device (e.g., a prosthetic heart valve). In FIG. 4A, the frame 220 is in a radially compressed configuration, which may be the configuration of the prosthetic device when it is mounted on a delivery apparatus, such as shown in FIG. 2. In FIG. 4B, the frame 220 is in a radially expanded configuration, which may be the configuration of the prosthetic device when it is radially expanded and implanted at the implantation site in a patient (e.g., the configuration of the prosthetic heart valve 10 shown in FIG. 1). Although the frame 220 is shown schematically in FIGS. 4A-4B, the frame can have any of various configurations, such as the frame 12 of FIG. 1, or any of various frames that are balloon-expandable, self-expandable or mechanically expandable, such as disclosed in the applications referenced above. Also, the prosthetic device 250 can be a prosthetic valve comprising one or more of the components described above for prosthetic valve 10, including leaflets 40 and inner skirt 16. In some examples, the prosthetic device 250 can be the prosthetic valve 10, except that the outer skirt 18 is replaced with the outer skirt 200.

The outer skirt 200 can comprise a base material 204 (which can also be referred to as a base layer or sealing layer). The base material 204 of the outer skirt 200 and the other skirts or coverings described herein can comprise various synthetic materials, including fabrics (e.g., polyethylene terephthalate fabric (PET) fabric) or ultra high molecular weight polyethylene (UHMWPE) fabric)), polytetrafluoroethylene (PTFE), thermoplastic polyurethane (TPU), a hybrid material comprising one or more fabric or polymeric materials (e.g., PET coated in TPU), or natural tissue (e.g., pericardial tissue). In some examples, the base material 204 of the outer skirt 200 can comprise a woven material (such as woven PET) formed by weaving together two or more sets of woven fibers, strands, or yarns.

The reinforcement member 202 can be formed from one or more of a suture, yarn, a chord, or a wire (e.g., a shape memory wire such as a Nitinol wire) and attached to the base material 204 (also referred to as “the material”) of the outer skirt 200. In other examples, the reinforcement member 202 can be a self-expandable stent or ring, such as made from Nitinol, which, for example, can be formed by laser cutting the reinforcement member from a metal tube or other techniques known in the art for forming stents. Thus, the reinforcement member 202 can be supported by the base material 204 of the outer skirt 200. In some examples, the reinforcement member 202 can embedded within or woven into the base material 204 of the outer skirt. For example, when the base material 204 comprises a weave of fibers, the sutures, yarns, chords, or wires making up the reinforcement member 202 can be woven into the weave of fibers making up the base material 204. In alternate examples, the reinforcement member 202 can be attached to the base material 204 by one or more fasteners, such as sutures, or an adhesive. In such examples, the reinforcement member 202 can be secured to an inner surface of the base material 204 or an outer surface of the base material.

The outer skirt 200 can comprise opposing first and second edge portions 206, 208 (which can also be referred to as short edges or edge portions) which each extend between an outflow edge portion 210 (also referred to herein as an “upper edge portion”) and an inflow edge portion 212 (also referred to herein as a “lower edge portion”) of the outer skirt 200.

Though the first and second edge portions 206, 208 are depicted in FIG. 3 as being perpendicular to the inflow edge portion 212, in alternate examples, the first and second edge portions 206, 208 can be non-perpendicular to the inflow edge portion 212. For example, in alternate examples, the first and second edge portions 206, 208 can extend at angles of about 45 degrees (or in a range of 40 to 50 degrees) relative to the inflow edge portion 212. Thus, an overall general shape of the outer skirt 200 can be that of a rhomboid or parallelogram, or a rectangle (as shown in FIG. 3).

When the outer skirt 200 is converted into its annular configuration (e.g., when mounted around a frame of a prosthetic device, as shown in FIGS. 4A and 4B), the first and second edge portions 206, 208 can be disposed adjacent one another or overlap one another, and can then be secured together via one or more fasteners (e.g., sutures).

The outflow edge portion 210 of the outer skirt 200 can be is disposed closer to an outflow end 222 of a frame 220 of the prosthetic device than the inflow edge portion 212 when the outer skirt 200 is arranged around the frame 220 of the prosthetic device, as shown in FIGS. 4A and 4B. FIGS. 3-4B depict the outflow edge portion 210 being relatively straight and parallel to the inflow edge portion 212. However, in alternate examples, the outflow edge portion 210 can have an alternate shape, such as being formed with a plurality of projections that define an undulating shape that generally follows the shape or contour of the struts of the frame to which the outflow edge portion 210 is secured.

The inflow edge portion 212 of the outer skirt 200 can be disposed at an inflow end 224 of the frame 220 of the prosthetic device (FIGS. 4A and 4B). In this way, the outer skirt 200 can extend from the inflow end 224 of the frame 220 toward, but spaced away from, the outflow end 222 of the frame 220.

Both the outflow edge portion 210 and the inflow edge portion 212 of the outer skirt 200 can be secured (directly) to the frame 220 (e.g., to the struts of the frame via sutures, as shown in FIG. 1 for example) while a remainder of the outer skirt 200 that is disposed between the inflow edge portion 212 and the outflow edge portion 210 is unattached to the frame 220 (FIGS. 4A and 4B). In some examples, the reinforcement member 202 can be attached to an intermediate portion 214 (or middle portion) of the outer skirt 200 that is disposed between (and spaced away from) the inflow edge portion 212 and the outflow edge portion 210. Thus, the reinforcement member 202 can also be unattached from the frame 220 and free to extend radially outward and away from the frame 220, relative to a central longitudinal axis 226 of the frame 220.

The frame 220 desirably is configured to foreshorten when the frame is radially expanded from the radially compressed state (FIG. 4A) to the radially expanded state (FIG. 4B). Thus, when the frame is radially expanded, the outflow edge portion 210 and the inflow edge portion 212 can move closer together, thereby creating slack or excess material in the base material 204 between the inflow and outflow edge portions.

In some examples, the reinforcement member 202 can be referred to as a deformable reinforcement member 202 due to its ability to change or transform from a first shape or first configuration 230 when the frame 220 is in the radially compressed configuration (as shown in FIG. 4A) to a second shape or second configuration 232 when the frame 220 is in the radially expanded configuration (as shown in FIG. 4B).

In its first configuration 230 (FIG. 4A), the reinforcement member 202 can be elongated in an axial direction and disposed closer to the frame 220 in a radial direction (as compared to when in the second configuration 232). For example, when the reinforcement member 202 is in its first configuration 230, a height 216 (in the axial direction) of the reinforcement member 202 can be larger than when in the second configuration 232. In some examples, the height 216 of the reinforcement member 202 can be specified such that the reinforcement member 202 is spaced away from the outflow edge portion 210 and the inflow edge portion 212 in both the first configuration 230 and the second configuration 232.

In contrast, when the reinforcement member 202 is in the second configuration 232 (FIG. 4B), the height 216 of the reinforcement member 202 can be shorter than when in the first configuration 230. Further, in the second configuration 232 the reinforcement member 202 can extend or protrude radially outward and away from the outer surface of the frame 220, thereby causing the slack or excessive base material 204 along the intermediate portion 214 of the outer skirt 200 (to which the reinforcement member 202 is attached or embedded within) to extend or bulge radially outward from the frame 220 (FIG. 4B).

The ability of the reinforcement member 202 to change between the first configuration 230 and the second configuration 232 can be due to a material of the reinforcement member 202. For example, as introduced above, the reinforcement member 202 can comprise a stent, suture, braid, or wire (or multiple sutures, braids, and/or wires integrated together), such as a shape-memory braid or wire (e.g., a Nitinol braid or wire), that can be configured to assume the first configuration 230 (FIG. 4A) when the frame 220 is radially compressed and the outer skirt 200 elongates axially with the frame 220 and transform into the second configuration 232 when the frame 220 is radially expanded and the outer skirt 200 shortens axially with the frame 220.

As an example, the reinforcement member 202 can be shape set to a shape that protrudes or bulges radially outward when the frame 220 is in the radially expanded configuration (FIG. 4B). This can be referred to as the “free state” of the reinforcement member 202. The material of the reinforcement member 202 can be configured such that the reinforcement member 202 can be deformed into its first configuration 230 when the frame is radially compressed. As introduced above, in this deformed, first configuration 230 the reinforcement member can elongate axially and press closer against the frame 220, thereby sandwiching or pressing the intermediate portion 214 of the outer skirt 200 against the outer surface of the frame 220. This can help to reduce an overall crimp profile of the radially compressed prosthetic device when radially compressed around a delivery apparatus (e.g., as shown in FIG. 2).

When the frame 220 is radially expanded (e.g., when being deployed by the delivery apparatus at the implantation site), the reinforcement member 202 can revert back to is free state, or the second configuration 232. In some examples, the reinforcement member 202 can be tuned to have a specified amount of radial strength and/or a specified amount of radial extension away from the frame 220. In this way, the reinforcement member 202 can have a more predictable and repeatable behavior by being made of a shape-memory material and tuning the behavior of the shape-memory material.

In some examples, as shown in FIGS. 3-4B, the reinforcement member 202 can comprise a circumferentially extending row of cells 234 arranged end-to-end around the outer skirt 200. Each cell 234 can have an axial height, which is the height 216 of the reinforcement member 202, and a width 218 (in the circumferential direction). The height 216 of each cell 234 can be longer in the first configuration 230 (FIG. 4A) and shorter in the second configuration 232 (FIG. 4B). Further, the width 218 of each cell 234 can be shorter in the first configuration 230 than in the second configuration 232. In the illustrated example, the cells 234 are four-sided, diamond-shaped cells. In other examples, the cells 234 can have any of various other shapes, such as hexagonal, oval, circular, or combinations thereof.

The reinforcement member 202 can have a thickness 228. The thickness 228 can be selected such that the reinforcement member 202 extends radially outward from the frame 220 of the prosthetic device 250 by a specified amount when the frame 220 is in the radially expanded configuration (FIG. 4B).

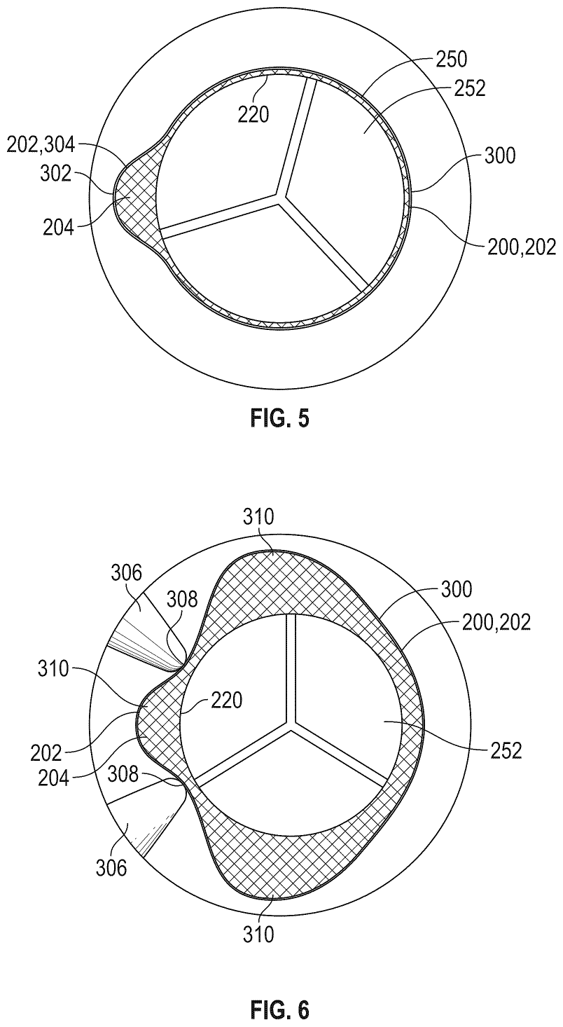

The reinforcement member 202 can also form a closed ring around a circumference of the outer skirt 200, as shown in FIGS. 4A and 4B. By being formed as a closed ring and from a deformable material (such as a metal wire, or a shape-memory material, as described above), the reinforcement member 202 can better fit the native anatomy (e.g., the native valve annulus) upon implantation. For example, as illustrated in FIGS. 5 and 6, the reinforcement member 202 can be configured such that when one region of the reinforcement member 202 is pressed radially inward by a portion of the native anatomy at the native valve annulus 300, adjacent regions of the reinforcement member 202 bulge or extend further radially outward (e.g., to maintain the outer perimeter of the ring of the reinforcement member 202). As shown in FIGS. 5 and 6, this allows the reinforcement member 202 to conform to the shape of the surrounding anatomy at the native valve annulus 300 (or alternate implantation site for the prosthetic device).

In FIGS. 5 and 6, the prosthetic device 250 is a prosthetic heart valve comprising the frame 220, leaflets 252 mounted inside the frame, and the outer skirt 200 disposed around the outer surface of the frame. Further, FIGS. 5 and 6 show a cross-section of the prosthetic heart valve taken along the middle portion 314, in a region of the reinforcement member 202.

Turning to FIG. 5, the prosthetic device 250 is implanted (radially expanded) within the native valve annulus 300, which includes an exemplary depression 302 therein. Such irregularities in the native valve annulus 300 can be caused by calcification of the native annulus. The portions of the native valve annulus 300 adjacent to the depression 302 can be narrower and press radially inward against the reinforcement member 202, thereby causing a portion or region 304 of the reinforcement member 202 not being pressed inward by the native valve annulus 300 to extend radially outward into the depression 302, allowing the outer skirt to seal against the native tissue within the depression. As a result, gaps between the prosthetic device 250 and the native valve annulus 300 can be reduced, thereby minimizing paravalvular leakage.

As another example, as shown in FIG. 6, the native valve annulus 300 may include one or more calcified nodules 306. As the one or more calcified nodules 306 press radially inward against first portions 308 (or first segments) of the reinforcement member 202, second portions 310 (or segments) of the reinforcement member 202 which are disposed adjacent to the first portions 308 bulge further radially outward to fill gaps in the native valve annulus 300 around the calcified nodules 306.

Thus, the reinforcement member 202 provides the prosthetic device 250 with improved perivalvular leakage sealing against the native anatomy without applying unnecessary forces against the calcified nodules 306.

In some examples, the reinforcement member 202 can comprise a plurality of circumferentially extending rows of cells. In some implementations, each cell of a row can be connected to one or more cells in an adjacent row. In other implementations, each row of cells can be spaced apart from each other in the axial direction (along the height of the base material 204. In this manner, each row of cells can be a separate reinforcement member coupled to the base layer.

In some examples, the reinforcement element 202 can have an alternate shape (other than an annular array of closed cells) extending circumferentially along (and around) the outer skirt, such as a zig-zag shape, a plurality of axially extending lines, circumferentially extending rings, or a plurality of circles, figure eights, triangles, or the like arranged end-to-end around the circumference of the outer skirt.

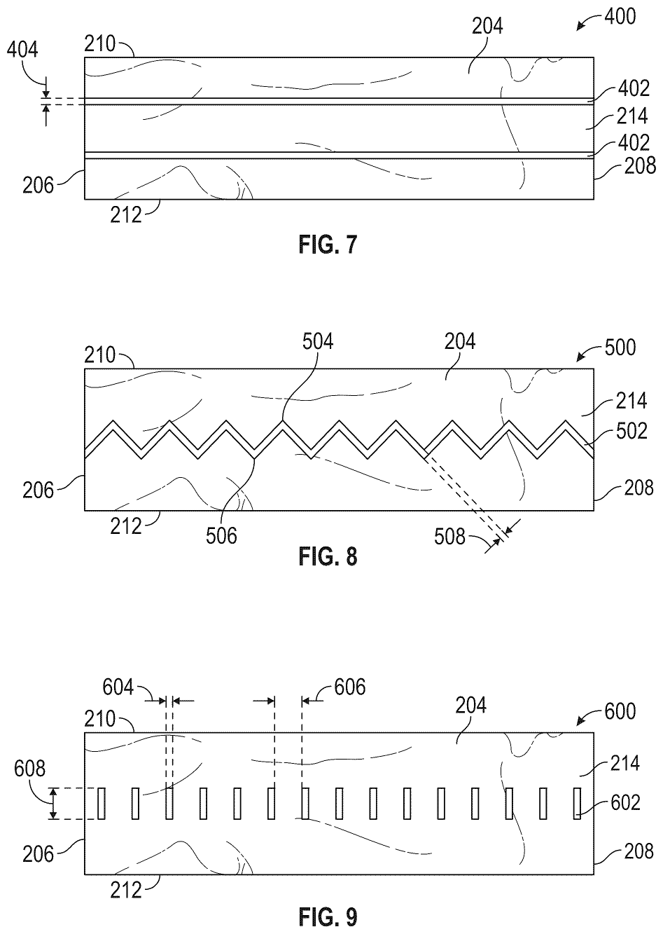

FIGS. 7-9 illustrate additional examples of reinforcement members for an outer skirt of a prosthetic device. The reinforcement members shown in FIGS. 7-9 can be similar to the reinforcement member 202 described above with reference to FIGS. 3-4B. Similar to the reinforcement member 202, the reinforcement members shown in FIGS. 7-9 can be attached to the base material 204 of the outer skirt, such as by suturing, weaving, an adhesive, or otherwise embedding the reinforcement member 202 within the base material 204. The outer skirts 400, 500, and 600 shown in FIGS. 7, 8, and 9, respectively, are shown in a flattened configuration off a frame of the prosthetic device. However, the outer skirts 400, 500, and 600 can be attached to a frame of a prosthetic device similar to as shown in FIGS. 4A and 4B for outer skirt 200.

Turning first to FIG. 7, the outer skirt 400 can include one or more reinforcement members 402 (two shown in the example of FIG. 7), each reinforcement member 402 shaped as a horizontally (or circumferentially once wrapped around the cylindrical frame of the prosthetic device) extending line or ring along the outer skirt 400. For example, as shown in FIG. 7, each reinforcement member 402 can be shaped as a line having a thickness 404. The thickness 404 can be selected such that each reinforcement member 402 extends radially outward from the frame of the prosthetic device to which the outer skirt 400 is attached when the frame is in a radially expanded configuration (e.g., similar to as shown in FIG. 4B).

In some examples, as shown in FIG. 7, each reinforcement member 402 can extend from the first edge portion 206 to the second edge portion 208 of the outer skirt 400 such that the reinforcement member 402 forms a ring around the outer skirt 400 when in its annular configuration around the frame of the prosthetic device.

In some examples, as shown in FIG. 7, the outer skirt 400 can comprise a plurality of reinforcement members 402 that are spaced apart from one another in the axial direction (e.g., two, three, four, five, or the like). Similar to the outer skirt 200, the reinforcement members 402 can disposed in an intermediate portion 214 of the outer skirt 400 and spaced away from the inflow edge portion 212 and the outflow edge portion 210.

FIG. 8 shows another exemplary outer skirt 500 comprising a reinforcement member 502 having an undulating shape, such as the illustrated a zig-zag pattern or a sinusoidal shape extending in a circumferential direction along the outer skirt 500. In some examples, the reinforcement member 502 can extend from the first edge portion 206 to the second edge portion 208 of the outer skirt 500, thereby forming a (closed) ring around the outer skirt 500 when in its annular configuration around a frame of a prosthetic device.

The undulating shape of the reinforcement member 502 can comprise a plurality of peaks 504 (or outflow apices) and a plurality of valleys 506 (or inflow apices) that are spaced away from the outflow edge portion 210 and the inflow edge portion 212 of the outer skirt 500, respectively.

A thickness 508 of the reinforcement member 502 can be selected such that the reinforcement member 502 extends radially outward from the frame of the prosthetic device to which the outer skirt 500 is attached when the frame is in a radially expanded configuration (e.g., similar to as shown in FIG. 4B).

FIG. 9 shows another exemplary outer skirt 600 comprising a plurality of reinforcement member 602 spaced apart from one another in the circumferential direction along the outer skirt 600, from the first edge portion 206 to the second edge portion 208 of the outer skirt 600. Each reinforcement member 602 can be shaped as a vertical line or axially extending rectangle having a thickness 604. In alternate examples, each reinforcement member 602 can be configured as an axially extending member having an alternate shape, such as oblong, curved, or s-shaped, with its longest dimension being in the axial direction.

The thickness 604 of the reinforcement members 602 can be selected such that the reinforcement members 602 extend radially outward from the frame of the prosthetic device to which the outer skirt 500 is attached when the frame is in a radially expanded configuration (e.g., similar to as shown in FIG. 4B). Further, a spacing 606 (in the circumferential direction) between adjacent reinforcement members 602 can be selected such that the base material 204 of the outer skirt 600 at the intermediate portion 214 bulges radially outward, all the way around the circumference of the outer skirt 600, when the prosthetic device is in the radially expanded configuration.

A height 608 (defined in the axial direction) of each reinforcement member 602 can be selected such that ends of each reinforcement member 602 are spaced away from the inflow edge portion 212 and the outflow edge portion 210 of the outer skirt 610 and each reinforcement member 602 is disposed within the intermediate portion 214 of the outer skirt 600. In other examples, one or more of the reinforcement members 602 can extend the entire height of the outer skirt from the inflow edge to the outflow edge.

In this way, by having one or more deformable reinforcement member arranged circumferentially along an outer skirt, a prosthetic device (such as a prosthetic heart valve) can better conform to an irregular geometry at an implantation site (such as a native valve annulus), thereby providing improved sealing against the native anatomy. In some examples, by being formed as a closed ring, portions of the reinforcement member can extend radially outward into depressions in the native valve annulus as other portions of the reinforcement member are pressed radially inward by protrusions in the native valve annulus. Further still, in examples where the reinforcement member is formed from a shape memory material (e.g., wire), the reinforcement member of the outer skirt can be configured to elongate axially and be disposed closer to the frame of the prosthetic device when the prosthetic device is in a radially compressed configuration and then extend radially outward from the frame when the prosthetic device is in a radially expanded configuration. As a result, the outer skirt of the prosthetic device can provide increased perivalvular leakage sealing while also reducing an overall crimp profile of the prosthetic device (e.g., when mounted around a delivery apparatus).

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: an annular frame; and an outer skirt disposed around an outer surface of the frame, the outer skirt comprising: a sealing layer comprising an outflow edge portion secured to the frame and an inflow edge portion secured to the frame; and a deformable reinforcement member attached to the sealing layer, the reinforcement member extending circumferentially along the sealing layer, wherein the reinforcement member is configured to extend radially outward from the frame such that the sealing layer bulges radially outward and away from the frame when the frame is radially expanded from a radially compressed state to a radially expanded state, and wherein the reinforcement member comprises a circumferentially extending row of cells or an undulating shape.

Example 2. The prosthetic heart valve of any example herein, particularly example 1, wherein the reinforcement member comprises a shape-memory material.

Example 3. The prosthetic heart valve of any example herein, particularly either example 1 or example 2, wherein the reinforcement member comprises a Nitnol braid or Nitinol wire.

Example 4. The prosthetic heart valve of any example herein, particularly any one of examples 1-3, wherein the sealing layer comprises a fabric.

Example 5. The prosthetic heart valve of any example herein, particularly any one of examples 1-4, wherein the reinforcement member is woven into the sealing layer.

Example 6. The prosthetic heart valve of any example herein, particularly any one of examples 1-5, wherein the reinforcement member forms a closed ring around a circumference of the outer skirt.

Example 7. The prosthetic heart valve of any example herein, particularly any one of examples 1-6, wherein the reinforcement member is spaced away from the outflow edge portion and the inflow edge portion of the sealing layer.

Example 8. The prosthetic heart valve of any example herein, particularly any one of examples 1-7, wherein the reinforcement member is unattached to and spaced radially away from the outer surface of the frame.

Example 9. The prosthetic heart valve of any example herein, particularly any one of examples 1-8, wherein the reinforcement member comprises a circumferentially extending row of diamond shaped cells arranged end-to-end around the outer skirt.

Example 10. The prosthetic heart valve of any example herein, particularly any one of examples 1-8, wherein the reinforcement member is sutured to the sealing layer.

Example 11. The prosthetic heart valve of any example herein, particularly any one of examples 1-8, wherein the reinforcement member forms a zig-zag pattern around the outer skirt, and wherein peaks and valleys of the reinforcement member are spaced away from the outflow and inflow edge portions of the sealing layer.

Example 12. The prosthetic heart valve of any example herein, particularly any one of example 1-8, wherein the inflow and outflow edge portions of the sealing layer move closer together when the frame is radially expanded to form slack along an intermediate portion of the sealing layer.

Example 13. The prosthetic heart valve of any example herein, particularly any one of examples 1-12, further comprising a plurality of leaflets arranged within an interior of the frame.

Example 14. A prosthetic heart valve comprising: a radially expandable and compressible annular frame configured to move between a radially compressed configuration and a radially expanded configuration; and an outer skirt disposed around an outer surface of the frame, the outer skirt comprising: a sealing layer comprising an outflow edge portion secured to the frame and an inflow edge portion secured to an inflow end of the frame; and a reinforcement member coupled to the sealing layer and extending circumferentially along the sealing layer at an axial location that is between the outflow edge portion and the inflow edge portion, wherein the reinforcement member comprises a shape-memory material and is moveable between a first shape and a second shape, wherein in the first shape the reinforcement member is lengthened in an axial direction and disposed closer to the frame relative to the second shape, and wherein in second shape the reinforcement member is shortened in an axial direction relative to the first shape and protrudes radially outward and away from the frame.

Example 15. The prosthetic heart valve of any example herein, particularly example 14, wherein when the frame is in the radially compressed configuration the reinforcement member is in the first shape, and wherein when the frame is in the radially expanded configuration the reinforcement member is in the second shape.

Example 16. The prosthetic heart valve of any example herein, particularly either example 14 or example 15, wherein the sealing layer comprises a fabric and the reinforcement member is woven into the fabric.

Example 17. The prosthetic heart valve of any example herein, particularly any one of examples 14-16, wherein the reinforcement member comprises Nitinol.

Example 18. The prosthetic heart valve of any example herein, particularly any one of examples 14-17, wherein the first shape is a deformed configuration of the reinforcement member and the second shape is a free configuration of the reinforcement member.

Example 19. The prosthetic heart valve of any example herein, particularly any one of examples 14-18, wherein the reinforcement member forms a ring around a circumference of the sealing layer.

Example 20. The prosthetic heart valve of any example herein, particularly any one of examples 14-19, wherein the reinforcement member comprises a circumferentially extending row of cells arranged end-to-end around the outer skirt.

Example 21. The prosthetic heart valve of any example herein, particularly example 20, wherein a width of each cell is larger in the second shape than in the first shape.

Example 22. The prosthetic heart valve of any example herein, particularly any one of examples 14-19, wherein the reinforcement member comprises a plurality of rings around a circumference of the outer skirt that are spaced axially apart from one another.

Example 23. The prosthetic heart valve of any example herein, particularly any one of examples 14-19, wherein the reinforcement member forms a zig-zag pattern around the outer skirt, and wherein peaks and valleys of the reinforcement member are spaced away from the outflow and inflow edge portions of the outer skirt.

Example 24. The prosthetic heart valve of any example herein, particularly any one of examples 14-19, wherein the reinforcement member comprises a plurality of axially extending members that are spaced circumferentially apart from one another around the outer skirt, and wherein ends of each axially extending member are spaced away from the outflow and inflow edge portions of the outer skirt.

Example 25. The prosthetic heart valve of any example herein, particularly any one of examples 14-24, further comprising a plurality of leaflets arranged within an interior of the frame.

Example 26. The prosthetic heart valve of any example herein, particularly any one of examples 14-25, wherein the entirety of the reinforcement member is spaced radially away from the frame when the reinforcement member is in the second shape.

Example 27. A prosthetic heart valve comprising: an annular frame; and an outer skirt disposed around an outer surface of the frame, the outer skirt comprising: a sealing layer disposed around an outer surface of the frame; and a deformable reinforcement member coupled to the sealing layer, the reinforcement member extending circumferentially along the sealing layer and comprising a circumferentially extending row of cells.

Example 28. The prosthetic heart valve of any example herein, particularly example 27, wherein the cells are diamond shaped.

Example 29. The prosthetic heart valve of any example herein, particularly any one of examples 27-28, wherein the cells are arranged end-to-end with one another and form a closed ring around a circumference of the sealing layer.

Example 30. The prosthetic heart valve of any example herein, particularly any one of examples 27-29, wherein the reinforcement member is disposed in an intermediate portion of the sealing layer that is disposed between and spaced away from an outflow edge portion and an inflow edge portion of the sealing layer.

Example 31. The prosthetic heart valve of any example herein, particularly any one of examples 27-30, wherein the reinforcement member is unattached to the outer surface of the frame.

Example 32. The prosthetic heart valve of any example herein, particularly any one of examples 27-31, wherein the reinforcement member comprises a shape-memory material.

Example 33. The prosthetic heart valve of any example herein, particularly any one of examples 27-32, wherein the reinforcement member comprises a Nitnol braid, Nitinol wire, or a Nitinol stent.

Example 34. The prosthetic heart valve of any example herein, particularly any one of examples 27-33, wherein the sealing layer comprises a fabric.

Example 35. The prosthetic heart valve of any example herein, particularly example 34, wherein the reinforcement member is woven into the fabric.

Example 36. The prosthetic heart valve of any example herein, particularly any one of examples 27-35, wherein the reinforcement member and a portion of the sealing layer to which the reinforcement member is attached bulge radially outward and away from the frame when the frame is radially expanded.

Example 37. The prosthetic heart valve of any example herein, particularly any one of examples 27-36, further comprising a plurality of leaflets arranged within an interior of the frame.

Example 38. The prosthetic heart valve of any example herein, particularly any one of examples 27-37, wherein the frame comprises a plurality of interconnected struts that form open cells in the frame, and wherein the frame is configured to radially compress and expand between a radially compressed configuration and a radially expanded configuration.

Example 39. The prosthetic heart valve of any example herein, particularly any one of examples 27-38, wherein the sealing layer comprises inflow and outflow edge portions that are secured to the frame with sutures.

Example 40. A prosthetic heart valve comprising: an annular frame; and an outer skirt disposed around an outer surface of the frame, the outer skirt comprising: a sealing layer disposed around an outer surface of the frame; and a shape-memory reinforcement wire comprising a circumferentially extending row of cells supported by the sealing layer.

Example 41. The prosthetic heart valve of any example herein, particularly example 40, wherein the cells are diamond shaped.

Example 42. The prosthetic heart valve of any example herein, particularly any one of examples 40-41, wherein the cells are arranged end-to-end with one another and form a closed ring around a circumference of the outer skirt.

Example 43. The prosthetic heart valve of any example herein, particularly any one of examples 40-42, wherein the reinforcement wire is disposed in an intermediate portion of the sealing layer that is disposed between and spaced away from an outflow edge portion and an inflow edge portion of the sealing layer.

Example 44. The prosthetic heart valve of any example herein, particularly any one of examples 40-43, wherein the reinforcement wire is unattached to the outer surface of the frame.

Example 45. The prosthetic heart valve of any example herein, particularly any one of examples 40-44, wherein the sealing layer comprises excess material between an outflow edge portion and an inflow edge portion of the sealing layer, and wherein the material of the sealing layer to which the reinforcement wire is attached bulges radially outward and away from the frame with the reinforcement wire when the frame is radially expanded.

Example 46. The prosthetic heart valve of any example herein, particularly any one of examples 40-45, wherein the reinforcement wire comprises Nitinol.

Example 47. The prosthetic heart valve of any example herein, particularly any one of examples 40-46, wherein the sealing layer comprises a fabric.

Example 48. The prosthetic heart valve of any example herein, particularly example 47, wherein the reinforcement wire is woven into the fabric.

Example 49. The prosthetic heart valve of any example herein, particularly any one of examples 40-48, further comprising a plurality of leaflets arranged within an interior of the frame.

Example 50. The prosthetic heart valve of any example herein, particularly any one of examples 40-49, wherein the frame comprises a plurality of interconnected struts that form open cells in the frame, and wherein the frame is configured to radially compress and expand between a radially compressed configuration and a radially expanded configuration.

Example 51. A method comprising: radially expanding a prosthetic heart valve from a radially compressed configuration to a radially expanded configuration within a native valve annulus of a heart, wherein the prosthetic heart valve comprises an annular frame, a plurality of leaflets secured inside the frame, and an outer skirt disposed around an outer surface of the frame comprising a sealing layer secured to the frame at an outflow edge portion of the sealing layer and an inflow edge portion of the sealing layer; and during the radially expanding, transitioning a reinforcement member embedded within a portion of the sealing layer that is unattached to the frame from a first configuration to a second configuration, wherein in the first configuration the reinforcement member is disposed adjacent to the frame and is longer in an axial direction relative to the second configuration, and wherein in the second configuration the reinforcement member extends radially outward and away from the frame.

Example 52. The method of any example herein, particularly example 51, wherein the portion of the sealing layer that is unattached to the frame is a central portion disposed axially between the outflow edge portion and the inflow edge portion, and further comprising, during the radially expanding and as the reinforcement member transitions to the second configuration, bulging the central portion of the sealing layer radially outward such that it conforms to a shape of the native valve annulus.

Example 53. The method of any example herein, particularly example 52, further comprising, during the radially expanding, as a first segment of the reinforcement member and the central portion of the sealing layer is compressed radially inward by a protruding feature at the native valve annulus, radially extending a second segment of the reinforcement member and central portion of the sealing layer into a gap formed in the native valve annulus adjacent to the protruding feature.

Example 54. The method of any example herein, particularly either example 51 or example 52, further comprising, in response to compressing a first portion of the reinforcement member in a radially inward direction, extending an adjacent, second portion of the reinforcement member further radially outward.

Example 55. The method of any example herein, particularly any one of examples 51-54, wherein the reinforcement member comprises a circumferentially extending row of diamond shaped cells arranged end-to-end around the outer skirt, and wherein each diamond shaped cell is shorter in the axial direction and longer in a circumferential direction in the second configuration than in the first configuration.

Example 56. The method of any example herein, particularly any one of examples 51-55, wherein radially expanding the prosthetic heart valve includes shortening the sealing layer in the axial direction and expanding the sealing layer radially outward.

Example 57. A prosthetic heart valve comprising: an annular frame; and an outer skirt disposed around an outer surface of the frame, the outer skirt comprising: a sealing layer disposed around an outer surface of the frame; and a deformable reinforcement member coupled to the sealing layer, the reinforcement member extending circumferentially along the sealing layer and comprising a plurality of axially extending members that are spaced circumferentially apart from one another around the outer skirt.

Example 58. The prosthetic heart valve of any example herein, particularly example 57, wherein the sealing member includes an outflow edge portion secured to the frame and an inflow edge portion secured to the frame, and wherein ends of each axially extending member are spaced away from the outflow and inflow edge portions of the sealing layer.

Example 59. The prosthetic heart valve of any example herein, particularly either example 57 or example 58, wherein the reinforcement member is coupled to an intermediate portion of the sealing layer that is disposed between and spaced away from the outflow edge portion and the inflow edge portion of the sealing layer.

Example 60. The prosthetic heart valve of any example herein, particularly any one of examples 57-59, wherein the sealing layer comprises excess material between an outflow edge portion and an inflow edge portion of the sealing layer, and wherein the material of the sealing layer to which the reinforcement member is coupled bulges radially outward and away from the frame with the reinforcement member when the frame is radially expanded.

Example 61. The prosthetic heart valve of any example herein, particularly any one of examples 57-60, wherein the reinforcement member comprises Nitinol.

Example 62. The prosthetic heart valve of any example herein, particularly any one of examples 57-61, wherein the sealing layer comprises a fabric.

Example 63. The prosthetic heart valve of any example herein, particularly any one of examples 57-62, further comprising a plurality of leaflets arranged within an interior of the frame.

Example 64. The prosthetic heart valve of any example herein, particularly any one of examples 57-63, wherein the frame comprises a plurality of interconnected struts that form open cells in the frame, and wherein the frame is configured to radially compress and expand between a radially compressed configuration and a radially expanded configuration.

Example 65. A method comprising sterilizing the prosthetic heart valve, apparatus, and/or assembly of any example.

Example 66. A prosthetic heart valve of any one of examples 1-64, wherein the prosthetic heart valve is sterilized.

Example 67. The method of any one of examples 51-56, wherein the method is performed on a living animal or on a non-living simulation, such as on a cadaver, cadaver heart, anthropomorphic ghost, or simulator.

Example 68. A method of treating a heart on a simulation, wherein the method includes the method of any one of examples 51-56.

The features described herein with regard to any example can be combined with other features described in any one or more of the other examples, unless otherwise stated. For example, any one or more of the features of one skirt, covering, sealing member, or reinforcement member can be combined with any one or more features of another skirt, covering, sealing member, or reinforcement member. As another example, any one or more features of one prosthetic heart valve can be combined with any one or more features of another prosthetic heart valve.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A prosthetic heart valve comprising:
an annular frame; and
an outer skirt disposed around an outer surface of the frame, the outer skirt comprising:
a sealing layer comprising an outflow edge portion secured to the frame and an inflow edge portion secured to the frame; and
a deformable reinforcement member attached to the sealing layer, the reinforcement member extending circumferentially along the sealing layer, wherein the reinforcement member is configured to extend radially outward from the frame such that the sealing layer bulges radially outward and away from the frame when the frame is radially expanded from a radially compressed state to a radially expanded state, and wherein the reinforcement member comprises a circumferentially extending row of cells or an undulating shape.

2. The prosthetic heart valve of claim 1, wherein the reinforcement member comprises a shape-memory material.

3. The prosthetic heart valve of claim 1, wherein the reinforcement member comprises a Nitnol braid or Nitinol wire.

4. The prosthetic heart valve of claim 1, wherein the sealing layer comprises a fabric.

5. The prosthetic heart valve of claim 1, wherein the reinforcement member is woven into the sealing layer.

6. The prosthetic heart valve of claim 1, wherein the reinforcement member forms a closed ring around a circumference of the outer skirt.

7. The prosthetic heart valve of claim 1, wherein the reinforcement member is spaced away from the outflow edge portion and the inflow edge portion of the sealing layer.

8. The prosthetic heart valve of claim 1, wherein the reinforcement member is unattached to and spaced radially away from the outer surface of the frame.

9. The prosthetic heart valve of claim 1, wherein the reinforcement member comprises a circumferentially extending row of diamond shaped cells arranged end-to-end around the outer skirt.

10. A prosthetic heart valve comprising:
an annular frame; and
an outer skirt disposed around an outer surface of the frame, the outer skirt comprising:
a sealing layer disposed around an outer surface of the frame; and
a shape-memory reinforcement wire comprising a circumferentially extending row of cells supported by the sealing layer.

11. The prosthetic heart valve of claim 10, wherein the cells are diamond shaped.

12. The prosthetic heart valve of claim 10, wherein the cells are arranged end-to-end with one another and form a closed ring around a circumference of the outer skirt.

13. The prosthetic heart valve of claim 10, wherein the reinforcement wire is disposed in an intermediate portion of the sealing layer that is disposed between and spaced away from an outflow edge portion and an inflow edge portion of the sealing layer.

14. The prosthetic heart valve of claim 10, wherein the reinforcement wire is unattached to the outer surface of the frame.

15. The prosthetic heart valve of claim 10, wherein the sealing layer comprises excess material between an outflow edge portion and an inflow edge portion of the sealing layer, and wherein the material of the sealing layer to which the reinforcement wire is attached bulges radially outward and away from the frame with the reinforcement wire when the frame is radially expanded.

16. A method comprising:
radially expanding a prosthetic heart valve from a radially compressed configuration to a radially expanded configuration within a native valve annulus of a heart, wherein the prosthetic heart valve comprises an annular frame, a plurality of leaflets secured inside the frame, and an outer skirt disposed around an outer surface of the frame comprising a sealing layer secured to the frame at an outflow edge portion of the sealing layer and an inflow edge portion of the sealing layer; and
during the radially expanding, transitioning a reinforcement member embedded within a portion of the sealing layer that is unattached to the frame from a first configuration to a second configuration, wherein in the first configuration the reinforcement member is disposed adjacent to the frame and is longer in an axial direction relative to the second configuration, and wherein in the second configuration the reinforcement member extends radially outward and away from the frame.

17. The method of claim 16, wherein the portion of the sealing layer that is unattached to the frame is a central portion disposed axially between the outflow edge portion and the inflow edge portion, and further comprising, during the radially expanding and as the reinforcement member transitions to the second configuration, bulging the central portion of the sealing layer radially outward such that it conforms to a shape of the native valve annulus.

18. The method of claim 16, further comprising, in response to compressing a first portion of the reinforcement member in a radially inward direction, extending an adjacent, second portion of the reinforcement member further radially outward.

19. The method of claim 16, wherein the reinforcement member comprises a circumferentially extending row of diamond shaped cells arranged end-to-end around the outer skirt, and wherein each diamond shaped cell is shorter in the axial direction and longer in a circumferential direction in the second configuration than in the first configuration.

20. The method of claim 16, wherein radially expanding the prosthetic heart valve includes shortening the sealing layer in the axial direction and expanding the sealing layer radially outward.
